(19) United States
(12) Patent Application Publication
(10) Pub. No.: US 2007/0238129 A1
(43) Pub. Date: Oct. 11, 2007

(54) Catalytically inactive enzymes for affinity binding

(76) Inventor: Susanne C. Moyer, Newark, DE (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/392,132

(22) Filed: Mar. 29, 2006

(51) Int. Cl. G01N 33/53; C12N 9/16

(52) U.S. Cl. 435/7.1; 435/196; 530/352

(57) ABSTRACT

Provided is a catalytically-inactive, dual-specificity phosphatase (dsPTP) or peptidomimetic affinity capture molecule attached to a matrix, for binding a phosphorylated polypeptide. The invention further provides a method for removing a phosphorylated polypeptide from a sample using the composition and kits useful in the practice of the method.

### FIELD OF THE INVENTION

The present invention relates generally to affinity chromatography. Specifically, the invention is a composition to isolate a phosphorylated polypeptide using a catalytically inactive protein from the family of dual-specificity phosphatases. Methods of using the composition are provided.

### BACKGROUND

Phosphorylation of proteins is a well-known, biologically significant, post-translational modification. Amino acids having a side chain with a hydroxyl group, namely serine, threonine and tyrosine, are the typical sites of phosphorylation. The phosphorylation/dephosphorylation cycle, comprising specific kinases and phosphatases, has long been recognized as a critical part of many signaling pathways. See, e.g., Greengard, Science 199:146-152 (1976) and Tonks et al., Curr. Opin. Cell Biol. 113:182-195 (2001). Not unexpectedly, abnormalities in the phosphorylation/dephosphorylation cycle have been found to play a role in numerous diseases, ranging from immune deficiencies to cancer. For instance, phosphatase Cdc25A, normally involved in cell-cycle regulation by removing inhibitory phosphates from particular kinases, is overexpressed in primary breast cancers (Cangi et al., J. Clin. Invest. 106:753-761 (2000)). It is also believed that phosphorylation is altered during aging. See, e.g., “Protein Phosphorylation in Aging and Aging-Related Diseases” (Mattson, ed.) In: Advances in Cell Aging and Gerontology 16, Elsevier, New York, N.Y., 2004.

The isolation of phosphorylated proteins, therefore, is of great interest. Methods used to isolate phosphorylated proteins include ion exchange, selective modification, immobilized metal affinity chromatography and immunoaffinity. Anion exchange is not specific and will bind any acidic protein. Therefore, it is not specific to the isolation of only phosphorylated proteins.

Selective modification involves biotinylating phosphoserine (pSer) or phosphothreonine (pThr) on phosphorylated proteins, then capturing those proteins using avidin affinity (Oda et al., Nat. Biotech. 19:379-382 (2001)). This method, however, only works for phosphoseryl or phosphothreonyl residues. Thus, it is ineffective for the isolation of all phosphorylated proteins.

Immobilized metal affinity chromatography (IMAC) employs a metal chelating ligand covalently immobilized to a solid phase support. A metal ion, known to have inherent affinity to a phosphorylated ligand is “loaded,” or chelated with the immobilized ligand and is used to interact and isolate the phosphorylated substrate. The general principle for this metal-substrate interaction is described in the principles of hard and soft acid and base theory, where phosphate is considered a hard acid, and the metal ions implemented are selected from those classified as hard bases. The primary shortcomings of this method are: 1) other components of proteins and peptides, namely carboxylates, are also considered hard acids, and thus, interact with the metal ions that are utilized; and 2) the phosphate-metal interaction is typically not strong enough to be used for the isolation of larger molecules, such as phosphoproteins. See, for instance, Posewitz et al., Anal. Chem. 71:2883-2892 (1999); Ueda et al., J. Chromatogr. A 988:1-23 (2003); U.S. Patent Publication No.2005/0054077.

Immunoaffinity-grade antibodies are available only for phosphotyrosine (pTyr), but not for the other phosphorylated proteins of interest. Unfortunately, antibodies that select for phosphoserine or phosphothreonine also recognize specific flanking amino acids. Therefore, they do not recognize all proteins having phosphoseryl and/or phosphothreonyl residues. Recent work by Comb and coworkers (Nat. Biotech. 2005, 23, 94-101; U. S. Pat. No. 6,441,140; U.S. Patent Publication No. 2003/0068652; U.S. Patent Publication No. 2003/0044848) describes the development of so-called motif-specific, context-independent antibodies, that bind to phosphorylation sites. It is disclosed that antibodies specific for phosphor-serine, threonine, and tyrosine, but not dependent on flanking amino acid residues, can be developed and used for the selective capture of phosphorylated peptides and proteins. The use of these antibodies for the isolation of phosphotyrosine-containing peptides was demonstrated. This technology is available as a kit from Cell Signaling Technology (Beverly, Mass.). However, the described technology has yet to be demonstrated for phosphoserine and phosphothreonine substrates.

Phosphatases are quite abundant. These enzymes bind to a phosphorylated site, and then the phospho-modification is hydrolyzed from the protein. For instance, there are over one hundred human genes that encode or are thought to encode protein tyrosine phosphatases (PTPs) (Alonso et al., Cell 117:699-711, (2004)). Phosphatases are classified into three groups based on specificity: 1) those that hydrolyze only phosphoserinyl and phosphothreonyl residues; 2) those that hydrolyze only phosphotyrosyl residues (the PTPs); and 3) those that recognize phosphotyrosyl residues, as well as phosphoseryl or phosphorthreonyl (dual-specificity phosphatases; dsPTPs). A few dsPTPs are, in fact, known to recognize all three phosphorylated residues. The signature catalytic motif in a PTP is HCXXGXXR(S/T). The cysteine in this motif is essential for catalysis. A phosphatase binds to a phosphorylated residue and forms a covalent intermediate between the catalytic cysteine and the phosphorylated residue. Hydrolysis of the covalent intermediate removes the phospho-modification from the residue.

A naturally-occurring catalytically inactive dsPTP, Sfb1, has been identified, where the catalytic cysteine residue was missing (Cui et al., Nat. Genet. 18:303-305 (1998) and De Vivo et al., Proc. Natl. Acad. Sci. USA 95:9471-9476 (1998)). This protein is enzymatically inactive, but is able to bind a phosphorylated substrate (phosphorylated serine and phosphorylated tyrosine), and protect these substrates from the activity of other phosphatases. This protein was dubbed an “antiphosphatase.”

Similarly, the protein STYX is a dual-specificity protein tyrosine phosphatase-like protein that has the same active site motif as a PTP, except that in place of the catalytic cysteine is a glycine (Wishart et al., J. Biol. Chem. 270:26782-26785 (1995)). Consequently, STYX is catalytically inactive. As a result, STYX binds to phosphoserine, phosphothreonine and phosphotyrosine, but because it is catalytically inactive, it cannot hydrolyze the phosphate.

Pils et al. (Mol. Biol. Evol. 21: 625-631 (2004)) searched for other occurrences of naturally-occurring inactive phosphatases by exploring the protein sequences for the whole genomes of Homo sapiens, Mus musculus, Fugu rubripes, Anopheles gambiae, Drosophila melanogaster, Ciona intestinalis, and Caenorhabditis elegans. Their analysis focused on amino acid positions that are involved in catalysis or binding, the positions being: Cys123 for nucleophilic attack of the phosphorous forming a thiophosphate intermediate, Arg129 for substrate binding, and Asp91, which acts as a general acid in dephosphorylation and a general base in the hydrolysis of the phosphoenzyme, with the second reaction supported by Ser130 (positions are numbered according to the sequence of 1VHR). A total of 24 and 23 dsPTPs were identified in Homo sapiens and Mus musculus, respectively, and 4 of each were identified as inactive based on one of the amino acid sites for catalysis or binding having a substitution that would render the dsPTP catalytically inactive.

U.S. Pat. No. 5,912,138 discloses protein tyrosine phosphatases having mutations at the site of a particular invariant aspartic acid, thereby inactivating the enzyme activity. The '138 patent discloses the use of the mutated PTPs to identify substrates of the phosphatase, and suggests therapeutic applications of the mutated PTPs.

U.S. Pat. No. 6,551,810 discloses particular mutants of the dual-specificity phosphatase DSP-10 for use as “substrate trapping mutants,” and their use for identifying specific substrates for DSP-10.

Nevertheless, until the present invention, there has remained an unmet need in the art for a method by which phosphorylated proteins and polypeptides are specifically isolated and enriched, which is critical to the identification of those phosphorylated polypeptides for that are present in only trace amounts in large volume biological samples. The instant invention meets that need.

### SUMMARY

The present invention provides a composition for isolating a phosphorylated polypeptide comprising an affinity capture molecule attached to a matrix, wherein the affinity capture molecule comprises a catalytically-inactive dual-specificity phosphatase (dsPTP), or a peptidomimetic of a catalytically inactive dsPTP. In one embodiment, the catalytically-inactive dsPTP is a naturally-occurring one. Catalytically-inactive dsPTPs useful in the invention include, but are not limited to, STYX and Sbf1, and variants thereof, wherein the variant has about the same binding affinity as the non-variant from which it is derived. The matrix can comprise a material selected from the group consisting of polystyrene, polysaccharide, agarose, cellulose, polyacrylamide, silica, and porous glass.

In one embodiment, the composition comprises a second affinity capture molecule that is different form the first affinity capture molecule. In another embodiment, the second affinity capture molecule is a second catalytically-inactive dsPTP.

The invention further provides a method of isolating a phosphorylated polypeptide. The method includes the steps of contacting a sample suspected of comprising a phosphorylated polypeptide with the composition of the invention under conditions allowing binding of the phosphorylated polypeptide to the affinity capture molecule to form bound phosphorylated polypeptide, separating material that did not bind from the bound phosphorylated polypeptide, and releasing the bound phosphorylated polypeptide from the affinity capture molecule, thereby isolating a phosphorylated polypeptide.

In one embodiment, the composition is part of a chromatography column, a spin column, a filter, or a microfluidic device. In another embodiment, the sample suspected of comprising a phosphorylated polypeptide is treated to remove highly abundant proteins. This embodiment is particularly useful for serum samples.

The invention further provides kits for isolating a phosphorylated polypeptide.

Additional features, objects and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

### DETAILED DESCRIPTION OF CERTAIN PREFERRED EMBODIMENTS

The present invention provides a composition and method useful for the affinity capture of phosphorylated polypeptides. In one embodiment, the composition comprises an affinity binding molecule attached to a matrix. The invention further provides a method of removing a phosphorylated polypeptide from a sample using compositions of the invention. In general, a sample suspected of comprising a phosphorylated polypeptide is contacted with the inventive composition to allow binding of any phosphorylated protein to the affinity capture molecule. The unbound material, from which the phosphorylated polypeptide has been subtracted, is then removed by washing or rinsing. Optionally, the method further comprises releasing the bound phosphorylated polypeptide from the composition, thereby isolating the phosphorylated polypeptide. A method of the invention may be used in an application in which isolation of a phosphorylated polypeptide from a sample is necessary or desired, including protein purifications for research or medical purposes, as well as for diagnostics and therapeutics. Similarly, a method of the invention may be used in an application in which a sample from which a phosphorylated polypeptide is subtracted is necessary or desired. In addition, the invention provides a kit and columns useful in practicing the methods of the invention.

Affinity Capture Molecule

In one aspect, the affinity capture molecule comprises a polypeptide that comprises a catalytically-inactive dsPTP. In one embodiment, the catalytically-inactive dsPTP is a naturally-occurring antiphosphatase, such as, but not limited to STYX and Sfb1, a variant thereof, wherein the polypeptide retains the binding activity of the corresponding non-variant STYX or Sfb1 and remains catalytically inactive. Notably, STYX possesses the ability to bind to phosphoseryl, phosphothreonyl and phosphotyrosyl residues, but does not catalyze the hydrolysis of the phosphate group (i.e., catalytically inactive). However, any catalytically-inactive dsPTP that possesses the ability to bind phosphotyrosine in addition to phosphoserine and/or phosphothreonine, or any combination thereof, may be utilized. In another embodiment of the invention, the affinity capture molecule comprises a catalytically-inactive dsPTP comprising SEQ ID NO. 7 in which position 2 is not a cysteine.

As used herein, “variant” refers to a polypeptide that is derived from a catalytically-inactive dsPTP polypeptide or that has a portion that has significant sequence identity with a catalytically-inactive dsPTP polypeptide. As used herein, “non-variant” refers to any naturally-occurring sequence of a catalytically-inactive dsPTP polypeptide from which a variant catalytically-inactive dsPTP is derived. As used herein, “catalytically inactive” refers to essentially non-detectable catalytic activity, measured using para-nitrophenyl phosphate (pNPP) as a substrate, see the protocol described in Wishart et al. supra, 1995. As used herein, a “portion” of a polypeptide means at least about 20 sequential amino acids of a polypeptide; it may also comprise at least about 50 sequential amino acids. It is understood that a portion of a polypeptide may include every amino acid of the polypeptide.

As used herein, “about the same binding activity,” when it refers to an affinity capture polypeptide that is a variant (i.e., the variant STYX or Sfb1 polypeptide) is defined as having a ratio of variant binding affinity to non-variant binding affinity for a given phospho-amino acid ranging between about 0.1 to about 10, when the binding affinities are measured under the same or matched conditions. For instance, if the binding affinity of a non-variant STYX polypeptide is 10⁻⁶ M for a specific phosphorylated peptide, any variant having a binding affinity between about 10⁻⁷ M to about 10⁻⁸ M for the same phosphorylated peptide has, for the purposes of this invention, about the same binding activity as the non-variant STYX.

In another aspect, the affinity capture molecule is a peptidomimetic of a catalytically-inactive dsPTP, having the about the same binding activity as the catalytically-inactive dsPTP it mimics (mimicked dsPTP). As used herein, a “peptidomimetic” is a compound containing non-peptidic structural elements that is capable of mimicking the biological action of a parent peptide. A peptidomimetic may or may not comprise peptide bonds. As used herein, “about the same binding activity,” when it refers to an affinity capture polypeptide that is a peptidomimetic, is defined as having a ratio of peptidomimetic binding affinity to mimicked parent dsPTP binding affinity for a given phospho-amino acid ranging between about 0.1 to about 10, when the binding affinities are measured under the same or matched conditions.

Binding affinity may be measured by any method known to the skilled artisan. A non-limiting example of a method of assessing binding affinity by measuring complex formation includes labeling a phosphorylated peptide with a detectable label, such as a radioactive or fluorescent moiety. The catalytically-inactive dsPTP, or variant thereof, is contacted with the labeled peptide to form complexes, and then the amount of complex formed is quantified. Other methods include monitoring fluorescence quenching to quantify complex formation, equilibrium dialysis and related methods known to those skilled in the art. Other methods useful in measuring binding include, e.g., chromatography, electrophoresis, gel filtration, absorption, fractionation, immunoassay, and combinations thereof. In conditions where the dissociation rate of the STYX/phosphorylated peptide complex is much greater than the rate of catalysis (e.g. catalytically inactive polypeptides), the Michaelis constant, Kₘ, may be used as a binding affinity.

In one embodiment of the present invention, the affinity capture molecule is a polypeptide comprising SEQ ID No. 1, which is the amino acid sequence of mouse STYX, or a variant thereof. In another embodiment, the affinity capture molecule is a polypeptide comprising SEQ ID No. 3, which is the amino acid sequence of human STYX, or a variant thereof. Homology between SEQ ID No. 1 and SEQ ID No. 3 is about 95% identical. In another embodiment, the affinity capture molecule is a polypeptide comprising SEQ ID No. 5, which is the amino acid sequence of human Sfb1.

A variant comprises N-terminal truncations, or C-terminal truncations, or a combination of both N-terminal and C-terminal truncations of a catalytically-inactive dsPTP polypeptide or is significantly homologous to a catalytically-inactive dsPTP polypeptide. For the purposes of the present invention, a variant that has significant homology to a catalytically-inactive dsPTP polypeptide is a polypeptide, either naturally occurring or the result of recombinant technology, that is at least about 80% identical to a catalytically-inactive dsPTP polypeptide, having about the same binding activity as the catalytically-inactive dsPTP polypeptide, and is catalytically inactive. As used herein, “naturally occurring” as applied to an object refers to an object that can be found in nature. For example, a naturally-occurring polypeptide sequence is one that can be isolated from a source in nature and which has not been intentionally modified by man. In one embodiment, the PTP catalytic motif of the variant does not contain the catalytic cysteine. As used herein, “PTP catalytic motif” refers to the amino acid sequence HCXXGXXR(S/T) (SEQ ID No. 7) and the “catalytic cysteine” refers to the cysteine at the second position in the PTP catalytic motif. Positions 119-127 in SEQ ID Nos. 1 and 3 are the PTP catalytic motif with glycine at position 120, in place of the catalytic cysteine. Positions 1420-1428 in SEQ ID No. 5 are the PTP catalytic motif with leucine at position 1421, in place of the catalytic cysteine

A variant may have a total of up to about 10, up to about 30, up to about 50, up about 100 or up to about 135 amino acids truncated from the catalytically-inactive dsPTP polypeptide. For significantly larger catalytically-inactive dsPTP, a variant may have much larger N— and C-terminal truncations to yield a variant comprising the domain identified as the dsPTP catalytic domain. The amino acids may be truncated from the N-terminal, the C-terminal, or a combination of both. For instance, one variant has 10 amino acids truncated from the N-terminal. A second variant has 4 amino acids truncated from the N-terminal and 6 amino acids truncated from the C-terminal. The second variant is considered to have a total of 10 amino acids truncated from the STYX polypeptide from which it is derived. A third variant has about 100 amino acids truncated from the N-terminal. Yet another variant has about 35 amino acids truncated from the C-terminal and about 100 amino acids truncated from the N-terminal of a STYX polypeptide, such as in SEQ ID Nos. 1 and 3. A fourth variant consists essentially of domain 3 of Sbf1. A truncation variant useful in the composition and methods of the invention must have about the same binding activity as the untruncated catalytically-inactive dsPTP polypeptide from which the variant is derived. The variant must also be catalytically inactive.

In one embodiment, the affinity capture molecule is a polypeptide that is about 80% identical to a sequence from a dsPTP (where a “model” dsPTP is 1VHR), where a residue necessary for the enzymatic phosphatase activity has been modified, rendering the protein enzymatically inactive. Residues that have been identified as necessary for catalysis, based on the numbering of 1VHR, are: Cys123 for nucleophilic attack of the phosphorous forming a thiophosphate intermediate, Arg129 for substrate binding, and Asp91, which acts as a general acid in dephosphorylation and a general base in the hydrolysis of the phosphoenzyme, with the second reaction supported by Ser130. The precise location of these catalytically necessary residues can be determined for other dsPTP and dsPTP-like proteins by performing multiple sequence alignments using a tool, such as ClustalW (www(dot)ebi(dot)ac(dot)uk/clustalw/).

In another embodiment, the affinity capture molecule is a polypeptide that is about 80% identical to a naturally-occurring, catalytically-inactive dsPTP. In a yet another embodiment of the invention, the affinity capture molecule is a polypeptide comprising a portion that is at least about 80% identical to SEQ ID No. 1 or SEQ ID No. 3, having about the same binding affinity as SEQ ID No.1 or SEQ ID No. 3, respectively, and wherein position 120 of SEQ ID No. 1 or SEQ ID No. 3 is not a cysteine. In still another embodiment, the relevant portion of the polypeptide is at least about 90% identical or about 95% identical to SEQ ID No.1 or SEQ ID No. 3, having about the same binding affinity as SEQ ID No. 1 or SEQ ID No. 3, respectively, and wherein position 120 of SEQ ID No.1 or SEQ ID No. 3 is not a cysteine.

In another embodiment of the invention, the affinity capture molecule is a polypeptide comprising a portion that is at least about 80% identical to SEQ ID No. 5, having about the same binding affinity as SEQ ID No. 5, and wherein position 1421 of SEQ ID No. 5 is not a cysteine. In another embodiment, the relevant portion of the polypeptide is at least about 90% identical or about 95% identical to SEQ ID No. 5, having about the same binding affinity as SEQ ID No. 5, and wherein position 1421 of SEQ ID No. 5 is not a cysteine.

In another embodiment, the affinity capture molecule is a polypeptide that comprises a portion that is at least about 80% identical to SEQ ID No. 1, having about the same binding affinity as SEQ ID No. 1, and wherein position 120 of SEQ ID No. 1 is not a cysteine, and position 126 is a lysine or an arginine. In yet another embodiment, the affinity capture molecule is a polypeptide that comprises a portion that is at least about 80% identical to SEQ ID No. 1, having the same binding affinity as SEQ ID No. 1, and wherein position 120 of SEQ ID No. 1 is not a cysteine, position 126 is a lysine or an arginine and position 89 is an aspartic acid.

In yet another embodiment, the affinity capture molecule is a polypeptide that comprises a portion that is at least about 80% identical to SEQ ID No. 1 and has the same binding affinity as SEQ ID No. 1, but wherein position 120 is a cysteine and position 89, an invariant aspartic acid in PTPs, is mutated to an alanine, glycine or another non-conservative amino acid. In this embodiment, the catalytic cysteine appears to enhance phosphorylated polypeptide binding through the formation of a covalent, reversible thiol-phosphate intermediate. By changing the amino acid identity at position 89, however, the dephosphorylation of the thiol-phosphate intermediate is prevented. By comparison to other dsPTPs, the important amino acid residues needed for catalysis in the STYX sequence are Cys120, Arg126, Asp89, and Ser127.

Variants may be prepared by any method known to the skilled artisan, including, but not limited to, site-specific mutagenesis, random mutagenesis, alanine-scanning mutagenesis, and other well-known molecular biology techniques. See, e.g., Sambrook et al., 2001, Molecular Cloning: A Laboratory Manual, Cold Spring Harbor Laboratory Press, Cold Spring Harbor, N.Y.; Ausubel et al., eds., 2005, Current Protocols in Molecular Biology, John Wiley & Sons, New York; and Gerhardt et al. eds.,1994, Methods for General and Molecular Bacteriology, American Society for Microbiology, Washington, D.C.

As used herein, “identity” is used synonymously with “homology.” “Homologous,” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5═ share 50% homology.

The determination of percent identity between two nucleotide or amino acid sequences may be accomplished, e.g., using a mathematical algorithm. For example, a mathematical algorithm useful for comparing two sequences is the algorithm of Karlin and Altschul (Proc. Natl. Acad. Sci. USA 87:2264-2268 (1990)), modified as in Karlin and Altschul (Proc. Natl. Acad. Sci. USA 90:5873-5877 (1993)). This algorithm is incorporated into the NBLAST and XBLAST programs of Altschul et al. (J. Mol. Biol. 215:403-410 (1990)), and is accessed, for example, at the National Center for Biotechnology Information (NCBI) world wide web site having the universal resource locator www(dot)ncbi(dot)nlm(dot)nih(dot)gov/BLAST/. BLAST nucleotide searches may be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches may be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST may be utilized as described in Altschul et al. (Nucleic Acids Res. 25:3389-3402 (1997)). Alternatively, PSI-Blast or PHI-Blast are used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) are used. See www(dot)ncbi(dot)nlm(dot)nih(dot)gov.

The percent sequence identity between two sequences is determined using techniques similar to those described above, with or without allowing gaps. In calculating percent identity, typically, exact matches are counted.

Structure-function studies of several phosphatases have been done. In particular, the crystal structure of 1VHR, a dsPTP, has been solved (Yuvaniyama et al., Science 272: 1328-1331 (1996)). 1VHR has extensive sequence similarity to STYX (Wishart et al., J. Biol. Chem. 270:26782-26785 (1995)). Barford et al., Science 263:1397-1404 (1994)) and Jia et al. (Science 268:1754-1758 (1995)) disclose the crystal structures of human PTP1b and PTP1b bound to a phosphotyrosine-containing peptide, respectively. 1VHR shares extensive sequence similarity with PTP1B. Consequently, the skilled artisan has extensive guidance, for instance, in identifying positions in the STYX sequence that are likely to tolerate mutation and retain STYX binding activity, and those that are not. For instance, these data suggest that His119 and Gly123 in SEQ ID No.1 are likely to be important in forming the correct structure of the PTP catalytic motif. These data also suggest that Asp89 and Arg126 in SEQ ID No. 1 are likely to be important for binding a phosphorylated substrate.

Similarly, the skilled artisan is guided in the extent and location of N-terminal and C-terminal truncations that retain STYX binding activity. Sequence alignment of 1VHR and SEQ ID No. 1 suggests that the final about 35 to about 40 residues at the C-terminal end of SEQ ID No. 1 may not be essential to structure and function of STYX. Amino acids that are involved in the proper folding and hydrophobic core of a protein generally tolerate conservative amino acid changes. Some conservative mutations to the hydrophobic core would be expected to increase the stability of the protein, which may be desirable. Amino acids that are on the surface of a protein are typically the most tolerant to amino acid changes, particularly with regard to folding and structure. The skilled artisan is familiar with amino acid substitutions that are generally considered conservative. Therefore, the skilled artisan has extensive guidance regarding which amino acid changes at what positions in a STYX protein are likely to be tolerated in terms of STYX structure and binding activity.

Modified catalytically-inactive dsPTP polypeptides are also suitable for use in the composition of the present invention. Modifications (which do not normally alter primary sequence) include in vivo, or ex vivo, chemical derivatization of a polypeptide, e.g., acetylation, or carboxylation. Also included are modifications of glycosylation, e.g., those made by modifying the glycosylation patterns of a polypeptide during its synthesis and processing or in further processing steps. Such processing includes, e.g., exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine. Modifications, such as crosslinking, to improve the structural stability of the affinity capture molecule are encompassed herein. Modifications to render the affinity capture molecule protease resistant are also encompassed herein. Such modification may also extend the activity and lifetime of the affinity capture molecule.

It will be appreciated, of course, that the polypeptides used as affinity capture molecules will likely incorporate amino acid residues which are modified without affecting activity. For example, the termini may be derivatized to include blocking groups, i.e. chemical substituents suitable to protect and/or stabilize the N— and C-termini from “undesirable degradation,” a term meant to encompass any type of enzymatic, chemical or biochemical breakdown of the compound at its termini, which is likely to affect the function of the compound, i.e. sequential degradation of the compound at a terminal end thereof. Other derivations may be useful in attaching the affinity capture molecule to a matrix.

Blocking groups include protecting groups conventionally used in the art of peptide chemistry which will not adversely affect the activities of the polypeptide. For example, suitable N-terminal blocking groups are introduced by alkylation or acylation of the N-terminus. Examples of suitable N-terminal blocking groups include C₁-C₅ branched or unbranched alkyl groups, acyl groups, such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups, such as methyl, ethyl and propyl, and amide-forming amino groups, such as primary amines (—NH₂), and mono- and di-alkylamino groups, such as methylamino, ethylamino, dimethylamino, diethylamino, methylethylamino and the like, are examples of C-terminal blocking groups. Descarboxylated amino acid analogues, such as agmatine, are also useful C-terminal blocking groups, and can be either coupled to the peptide's C-terminal residue, or used in place of it. Further, it will be appreciated that the free amino and carboxyl groups at the termini can be removed altogether from the polypeptide to yield desamino and descarboxylated forms thereof, without affect on polypeptide activity.

In the methods of the invention, if the affinity capture molecule is prepared, it is so prepared using standard methods known to the skilled artisan. Methods include in vitro peptide synthesis and biological means. Biological methods include, without limitation, expression of a catalytically-inactive dsPTP coding sequence in a host cell and in vitro translation systems.

As used herein, “peptide”, “polypeptide” and “protein” refer to polymers of amino acids. While a peptide may generally be considered shorter than a polypeptide, that is having fewer amino acids, there is no specific number of amino acids that define the two and none is implied herein. The same is true with regard to a polypeptide and a protein. Thus, these terms are used interchangeably herein.

Merrifield-type solid phase peptide synthesis may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., Ann. Rev. Biochem. 69:923-60 (2000)). A great advantage to the utilization of a synthetic peptide route is the ability to produce large amounts of peptides, even those that rarely occur naturally, with relatively high purities, i.e., purities sufficient for research, diagnostic or, even therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, N.Y. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both which methods are well-known by those of skill in the art.

Incorporation of N— and/or C— blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Biological preparation of the affinity capture molecule involves expression of a catalytically-inactive dsPTP gene or coding sequence. A naturally-occurring catalytically-inactive dsPTP polypeptide may be obtained from cells expressing an endogenous catalytically-inactive dsPTP gene. Such cells, optionally, may be genetically modified to overexpress an endogenous gene, using standard molecular biology techniques. Genetic modifications to aid in purification, such as the addition of a secretion peptide signal, or a 6-His tag, may also be made to an endogenous gene.

Biological preparation using an exogenous catalytically-inactive dsPTP coding sequence, or a coding sequence for a variant thereof, may also be done to generate the affinity capture molecule. DNA coding sequences useful for preparing the affinity capture molecule for the instant invention include, e.g., SEQ ID Nos. 2, 4 and 6, which encode the polypeptides of SEQ ID Nos. 1, 3 and 5, respectively. Neutral sequence variants of these sequences, based on the degeneracy of the genetic code, are also useful. Similarly, a DNA sequence encoding any polypeptide variant described elsewhere herein is useful for preparing the affinity capture molecule.

An expression cassette comprising such a coding sequence may be used to produce biologically a catalytically-inactive dsPTP polypeptide. By “expression cassette” is meant a nucleic acid molecule comprising a coding sequence of catalytically-inactive dsPTP, or a variant thereof, operably linked to promoter/regulatory sequences necessary for transcription and translation of the coding sequence. As used herein, the term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence. As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements, which are required for expression of the gene product. The promoter/regulatory sequence may, for example, be one which expresses the gene product in an inducible manner.

A “promoter” is defined as an array of nucleic acid control sequences that direct transcription. As used herein, a promoter typically includes necessary nucleic acid sequences near the start site of transcription, such as, in the case of certain RNA-polymerase-II-type promoters, a TATA element, enhancer, CCAAT box, SP-1 site, etc. As used herein, a promoter also optionally includes distal enhancer or repressor elements, which may be located as much as several thousand base pairs from the start site of transcription. Promoters often have an element that is responsive to transactivation by a DNA-binding moiety such as a polypeptide, e.g., a nuclear receptor, Gal4, the lac repressor and the like.

An “inducible” promoter is a nucleotide sequence which, when operably linked with a polynucleotide which encodes or specifies a gene product, causes the gene product to be produced in a living cell substantially only when an inducer which corresponds to the promoter is present in the cell. A “constitutive promoter” is a promoter which drives expression of a gene to which it is operably linked, in a constant manner in a cell. By way of example, promoters which drive expression of cellular housekeeping genes are considered to be constitutive promoters. A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

Vectors for expression cassettes and methods for the introduction of exogenous DNA into cells with concomitant expression of the exogenous DNA in the cells are described, for example, in Sambrook et al., supra, 2001; Ausubel et al., supra, 2004. Techniques for introducing vectors into target cells include, but are not limited to, electroporation, photoporation, calcium precipitation, fusion, transfection, lipofection, viral targeting and the like.

Any expression vector compatible with the expression of a catalytically-inactive dsPTP polypeptide in a host cell is suitable for use in the instant invention, and can be selected from the group consisting of a plasmid DNA, a viral vector, and a mammalian vector. Vectors may be episomal, or may be provided for integration into the target cell genome via homologous recombination or random integration. Viral vectors useful in the methods of the invention include, but are not limited to, cytomegalovirus vectors, adenovirus vectors and retrovirus vectors, such as MigRI, MMLC, HIV-2 and ALV.

The vector comprising the expression cassette, or a vector that is co-introduced with the expression vector, can comprise a marker gene. Marker genes are useful, for instance, to monitor transfection efficiencies. Marker genes include genes for selectable markers, including, but not limited to, G418, hygromycin, and methotrexate, and genes for detectable markers, including, but not limited to luciferase and GFP.

The catalytically-inactive dsPTP coding sequence contained in an expression cassette may, optionally, be fused in-frame to other coding sequences. For instance, the coding sequence of an epitope or other detectable tag may be included. Such tags are useful, for instance, to assist in the rapid purification of the encoded catalytically-inactive dsPTP polypeptide or variant thereof. An example of such a tag is a 6-His sequence. The fusion may be at either the N-terminal or the C-terminal of a catalytically-inactive dsPTP, provided the catalytically-inactive dsPTP binding activity is maintained.

In the context of an expression vector, the vector may be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., supra, 2001 and Ausubel et al., supra, 2005.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems are well known in the art.

To ensure that the polypeptide obtained from either chemical or biological synthetic techniques is the desired polypeptide, analysis of the polypeptide composition should be conducted. Such amino acid composition analysis may be conducted using high resolution mass spectrometry to determine the molecular weight of the peptide. Alternatively, or additionally, the amino acid content of the peptide may be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to definitively determine the sequence of the peptide.

Prior to its use as an affinity capture molecule, the polypeptide is purified to remove contaminants. Any one of a number of a conventional purification procedures may be used to attain the required level of purity including, for example, reversed-phase high-pressure liquid chromatography (HPLC) using an alkylated silica column, such as C₄-,C₈- or C₁₈-silica, or variations thereof. A gradient mobile phase of increasing organic content is generally used to achieve purification, for example, acetonitrile in an aqueous buffer, usually containing a small amount of trifluoroacetic acid. Ion-exchange chromatography may be also used to separate polypeptides based on their charge. Gel filtration chromatography may be used to separate polypeptides based on their size.

Matrix

Although attachment is not required, the affinity capture molecule is useful when attached to a matrix in the composition of the instant invention. As used herein, “matrix” refers to a material providing structural support. “Matrix” is used interchangeably with “stationary phase” and “solid phase” herein. Materials useful as a matrix, include, but are not limited to, agarose, silica, including alkylated silica column, such as C₄-,C₈- or C₁₈-silica, or variations thereof, as used in HPLC, porous glass, polystyrene, polysaccharide, cellulose, polyacrylamide, Sepharose® (GE Healthcare, Piscataway, N.J.), Sepharose C L and other polymers. Materials may be crosslinked, such as crosslinked polystyrenes or crosslinked dextran, or otherwise modified. The matrix material is of any suitable physical shape, including, but not limited to, monoliths, columns, spheres, beads, and 2-dimensional chips or slides.

The affinity capture molecule is attached to the matrix covalently or non-covalently by any method known in the art, and include those matrices referenced by, e.g. Hermanson et al., Immobilized Affinity Ligand Techniques, Academic Press, Inc., CA (1992). Covalent attachment is preferred. In some embodiment, an affinity capture molecule is attached directly to an active group on the solid phase via a linker moiety. The linker moiety may be any type of molecule suitable for attachment, including, but not limited to, hydrocarbon chains and their derivatives, which may or may not possess other chemical elements, including, but not limited to: sulfur, oxygen, nitrogen, and phosphorous.

Active groups present on the solid phase that are used to covalently attach the affinity molecule include, but are not limited to, epoxide, carboxylate (via carbodiimide linkage to N-terminal), succinimide, thiol, and other active groups known to the skilled artisan. However, attachment chemistries that could interfere with residues necessary for phosphorylated polypeptide binding should be avoided. Covalent attachments useful in the composition include, but are not limited to, standard protein cross-linking chemistries, such as glutaraldehyde activation of amine-functionalized surfaces, trialkoxy aldehyde silanes, DMP (dimethyl pimelimidate), and N-hydroxysuccinimide active ester. Non-limiting examples of non-covalent attachments useful in preparing the composition of the invention include hydrophobic interactions and avidin/biotin systems.

In one embodiment of the present invention, the composition comprises a single type of affinity capture molecule. In another embodiment, the composition comprises two or more types of affinity capture molecules. For instance, in one example, two or more STYX polypeptides are employed in the composition of the invention. In another embodiment, two or more different catalytically-inactive dsPTPs are employed in the composition. The affinity capture molecules may further comprise antibodies to one or more specific phospho-amino acids. The term “antibody,” as used herein, refers to an immunoglobulin molecule, which specifically binds to a specific epitope on an antigen. Antibodies include intact immunoglobulins derived from natural sources or from recombinant sources, and may be immunoreactive portions of intact immunoglobulins. Antibodies are typically tetramers of immunoglobulin molecules. The antibodies useful in the present invention exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., Proc. Natl. Acad. Sci. USA 85:5879-5883 (1998); Bird et al., Science 242:423-426 (1988)). Also useful are Affibody® molecules, which are highly specific affinity proteins that can be designed to bind to any desired target protein (U.S. Pat. No. 5,831,012).

In embodiments comprising two or more different affinity capture molecules, the different affinity capture molecules may be mixed together first and, the mixture then bound to the solid phase to form a generic solid-phase phosphate affinity matrix. Alternately, the affinity capture molecules can be individually bound to the solid phase, and the resulting immobilized affinity molecules may be mixed in a desired ratio into a column or spin tube format to perform optimal capture of phosphorylated substrates (pTyr, pThr, and pSer). This mixed batch mode has been utilized in other affinity chromatographic methods for the capture of multiple substrates, and may be used to enhance the number of phosphorylated substrates captured by this method.

The composition of the invention is intended for placement in variety of devices useful for isolations. Such devices include, but are not limited to, monoliths, monolithic columns, chromatography columns, including HPLC columns, spin columns, filters, or microfluidic devices, such as Lab-on-a-chip technology and HPLC chip technology (Agilent Technologies, Palo Alto, Calif.), used for nanoflow HPLC analysis.

The composition of the invention is useful in any method of isolating a phosphorylated polypeptide. As used herein, a “phosphorylated polypeptide” refers to a polypeptide having one or more phosphoserines, phosphothreonines or phosphotyrosines, or any combination thereof, in its amino acid sequence. “Isolating a phosphorylated polypeptide,” as used herein, refers to enriching a population of polypeptides for the selected phosphorylated polypeptides of interest.

Affinity chromatography methods are well known to the skilled artisan. See, e.g., Dean, et al., (eds., Affinity Chromatography: A Practical Approach, IRL Press, Oxford, UK (1985) and Dechow, Affinity Chromatography in: Separation and Purification Techniques in Biotechnology, Noyes Publications, Park Ridge, N.J. (1989) pages 416-484). Affinity chromatography is useful in many different applications, including diagnostic, industrial and research applications. Affinity chromatography is also useful in proteomics (see, e.g., Lee et al, Anal. Chem. 324:1-10 (2004)).

In brief, the method of the invention involves contacting a sample suspected of comprising a phosphorylated polypeptide with the inventive composition under conditions allowing binding of the phosphorylated polypeptide of interest to the affinity capture molecule. The non-bound material is then separated from the bound material by washing. In one embodiment, the non-bound material, from which phosphorylated polypeptides have been subtracted, is collected for subsequent use. In another embodiment, the bound phosphorylated polypeptide is released from the affinity capture molecule, thereby providing an isolated phosphorylated polypeptide product.

Buffers used in the methods of the invention may comprise one or more compounds to inhibit proteolytic activity possibly present in the sample that might degrade the affinity capture molecules. Non-limiting examples of such compounds, include aprotin, leupeptin and DMSF. Such compounds may also added to a sample to prevent or inhibit proteolytic degradation of the desired phosphorylated polypeptides contained in the sample.

Any sample suspected of comprising a phosphorylated polypeptide are useful in the method of the invention, including biological samples. Such biological samples include, but are not limited to, body fluids, such as blood, serum, plasma, saliva, semen and urine, and the like, and tissues. Tissue samples are typically treated so as to remove connective tissue and the cells are lysed, or otherwise treated to release the phosphorylated polypeptides.

In one embodiment, highly abundant proteins are removed, or significantly reduced in quantity, from a biological sample prior to isolating a phosphorylated polypeptide according to the method of the invention. As used herein, “highly abundant proteins” refer to a group of proteins comprising about 40% or more (by weight) of the total protein in a sample; although they may comprise 50% or more; or 60% or more; or 70% or more, and often as much as 80% or more (by weight) of the total protein in a sample.

For instance, in human serum, highly abundant proteins include albumin, IgG, IgA, haptoglobin, transferrin and antitrypsin, which together can represent more than 70% of the total sample. Such highly abundant proteins may be removed by any of a number of methods known to the skilled artisan. For instance, highly abundant proteins in a sample of serum may be removed using affinity chromatography, such as, but not limited to, Agilent's Multiple Affinity Removal System. Other methods to remove highly abundant proteins include immunodepletion and selective fractionation. Once highly abundant proteins are removed, one is left with a sample that, for instance, comprises only ˜25% of the original sample, but also only a small percentage of the original protein material. As a result, a phosphorylated protein representing only 0.001% of the original protein material is now at least 4-fold enriched in the remaining sample without the highly abundant proteins present. Because of the highly sensitive affinity of the composition of the present invention it is now possible to expressly select and isolate such a rarely occurring phosphorylated protein in very small sample volume, such as that remaining after the removal of the highly abundant proteins.

In an alternative embodiment, a biological fluid or tissue sample is obtained from a subject suspected of having, or diagnosed with, or one with a predisposed susceptibility to a disease or disorder involving an aberration in a particular phosphorylation/dephosphorylation system. Such sample is then compared with a normal standard or control sample from a normal subject to determine an increased or decreased presence of the phosphorylated protein of interest. In one embodiment, the highly abundant proteins are removed from the sample prior to affinity binding to the composition of the invention.

The composition and method of the invention are also useful for removing phosphorylated polypeptides from in vitro cell culture samples. In one embodiment, the phosphorylated polypeptides are isolated.

Following chromatographic separation according to the method of the invention, the resulting phosphorylated polypeptides may be further evaluated using well known analytical methods, such as SDS-PAGE, 2D electrophoresis, capillary electrophoresis (CE), UV-VIS absorption spectroscopy, fluorescence spectroscopy, thin-layer chromatography, mass spectrometry, protein fingerprinting methods, proteomic analyses, and so on. See also Hunter et al., eds., “Protein Phosphorylation, Part B: Analysis of Protein Phosphorylation, Protein Kinase Inhibitors, and Protein Phosphatases, Volume 201” in: Methods in Enzymology, Academic Press, New York, N.Y. (1991). Such isolated phosphorylated polypeptides may then be used for therapeutic and other purposes.

The invention further provides a kit useful for isolating a phosphorylated polypeptide comprising the composition of the invention and an instructional material. As used herein, an “instructional material” includes a publication, a recording, a diagram, or any other medium of expression which can be used to communicate the usefulness of the inventive composition for its designated use. The instructional material of the kit of the invention may, for example, be affixed to a container which contains the composition or be shipped together with a container which contains the composition. In one embodiment of the invention, the composition is positioned in a chromatography device, such as chromatography column. Particularly useful in the invention are such columns of a size commensurate with passage of volume less than 5 ml, or less than 1 ml or less than 0.1 ml or less than 0.01 ml or even smaller volumes.

Chromatography columns are produced in a variety of dimensions, which are based on the application for which the column is to be used. According to an embodiment of the invention, column dimension may range from about 0.05 mm to about 21.2 mm in inner diameter and from about 5 mm to about 250 mm in length. According to an embodiment of the invention column inner diameters may be from about 0.05 mm to about 10 mm. According to an embodiment of the invention column inner diameters may be from about 0.075 mm to about 5 mm. According to an embodiment of the invention column lengths range from 5 to 250 mm or from 20 mm to 150 mm. A chromatography column typically comprises an inlet and an outlet. In some embodiments, one or both of the inlet and the outlet comprise a filter and/or a frit.

The instructional material would be included with such a column. Alternatively, the instructional material may be shipped or provided separately from the container with the intention that the instructional material and the composition be used cooperatively by the recipient. Such instructional materials may also be provided via the Internet to accompany the container.

The present invention is further described in the following examples. These examples are provided for purposes of illustration only, and are not intended to be limiting unless otherwise specified. The various scenarios are relevant for many practical situations, and are intended to be merely exemplary to those skilled in the art. These examples are not to be construed as limiting the scope of the appended claims. Thus, the invention should in no way be construed as being limited to the following examples, but rather, should be construed to encompass any and all variations which become evident in light of the teaching provided herein.

### EXAMPLES

### Example 1

STYX protein expression is accomplished by methods known to those skilled in the art (see, e.g., Wishart et al., supra, 1995). The resulting expressed protein is purified by standard protein purification methods. The purified protein is attached to a solid phase matrix possessing an appropriate active group to yield an immobilized affinity reagent. Chemical attachment of the affinity molecule is accomplished by methods known to those skilled in the art.

The immobilized affinity reagent is contacted with a sample suspected of comprising phosphorylated polypeptides in a suitable binding buffer (20 mM Tris-HCl pH 8) to allow binding of any phosphorylated polypeptides to the affinity reagent. Following the binding, the affinity reagent is rinsed with additional volumes of binding buffer to remove any unbound molecules from the system. After washing, the bound phosphorylated material is disassociated from the immobilized affinity reagent by the use of a suitable elution buffer (such as 50 mM glycine-HCl pH 2.3 or 100 mM phenylphosphate in 20 mM Tris-HCl, pH 7.4). The pool of phosphorylated polypeptides is subjected to protein fingerprinting analysis to identify them.

The affinity reagent is reequilibrated and regenerated by repeated washing with binding buffer, in order to reuse the affinity reagent.

### Example 2

An affinity capture molecule comprising a polypeptide of SEQ ID No. 1 that is N-terminally and C-terminally truncated (having a total truncation of about 100 amino acids), is expressed in vitro, using a linked transcription/translation system. The polypeptide is purified and covalently attached to a solid phase matrix to produce an immobilized affinity reagent. The polypeptide is cross-linked to stabilize its structure. The immobilized affinity reagent is placed into a channel of a microfluidic device to serve as a separations device, such as an HPLC column.

### Example 3

An affinity capture molecule, which is a polypeptide of SEQ ID No. 1 that is N-terminally truncated by about 100 amino acids and C-terminally truncated by about 40 amino acids, is synthesized by a standard solid phase peptide synthesis process. The polypeptide is purified and covalently attached to a solid phase matrix to produce an immobilized affinity reagent. The immobilized affinity reagent is placed into a column of a microfluidic device.

A human serum sample is pretreated to remove highly abundant proteins. The immobilized affinity reagent is then contacted with the pretreated serum sample in a suitable buffer to allow binding of phosphorylated polypeptides to the affinity reagent. Following the binding, the affinity reagent is rinsed with additional volumes of binding buffer to remove any unbound molecules from the system. After washing, the bound phosphorylated material is disassociated from the immobilized affinity reagent by the use of a suitable elution buffer and subject to size separation and further analysis.

The affinity reagent is reequilibrated and regenerated by repeated washing with binding buffer, in order to reuse the affinity reagent.

### Example 4

To investigate phosphorylation sites on integral membrane proteins, HeLa cells are grown by standard methods. Cells are treated with 0.1 mM pervanadate. HeLa cell membranes are prepared by a modified carbonate treatment procedure to enrich the integral membrane fraction (Blonder et al., J. Prot. Res. 1(4):351-60 (2002); Fujiki et al., J. Cell Biol. 93 (1):97-102 (1982)). Integral membrane fractions are chemically digested with CNBr, followed by enzymatic digestion with trypsin, using standard methods. Resulting peptidic fragments are recovered, dried, resuspended in binding buffer (20 mM Tris, pH 7.4), and are allowed to interact with a phosphate binding column containing the composition of Example 1. Phosphopeptides are eluted with a suitable elution buffer, and are then analyzed by LC/MS.

The disclosures of each patent, patent application and publication cited or described in this document are hereby incorporated herein by reference, in their entirety.

While the foregoing specification has been described with regard to certain embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. A composition for binding a phosphorylated polypeptide, the composition comprising an affinity capture molecule attached to a matrix, wherein said affinity capture molecule comprises a catalytically-inactive dual-specificity phosphatase (dsPTP) or peptidomimetic thereof.

2. The composition of claim 1, wherein the catalytically-inactive dsPTP is naturally occurring.

3. The composition of claim 1, wherein the catalytically inactive dsPTP comprises a variant of SEQ ID NO. 7 in which position 2 is not a cysteine.

4. The composition of claim 1, wherein the catalytically-inactive dsPTP is selected from the group consisting of:
a polypeptide comprising SEQ ID No. 1;
a polypeptide comprising a portion that is at least about 80% identical to SEQ ID No. 1, wherein position 120 of SEQ ID No. 1 is not a cysteine;
a polypeptide comprising a portion that is at least about 80% identical to SEQ ID No. 1, wherein position 120 of SEQ ID No. 1 is a cysteine and position 89 of SEQ ID No. 1 is not an aspartic acid, and
a polypeptide comprising a variant of SEQ ID No. 1 that consists of an N-terminal truncation of SEQ ID No. 1, a C-terminal truncation of SEQ ID No. 1, and any combination thereof;
wherein the polypeptide has about the same binding affinity as SEQ ID No. 1.

5. The composition of claim 1, wherein the catalytically-inactive dsPTP is selected from the group consisting of:
a polypeptide comprising a portion that is at least about 80% identical to SEQ ID No.1, wherein position 120 of SEQ ID No.1 is not a cysteine and position 126 is a lysine or an argininine; and
a polypeptide comprising a portion that is at least about 80% identical to SEQ ID No.1, wherein position 120 of SEQ ID No.1 is not a cysteine, position 126 is a lysine or an arginine and position 89 is an alanine.

6. The composition of claim 1, wherein the catalytically-inactive dsPTP is a polypeptide comprising a portion that is at least about 90% identical to SEQ ID No. 1, wherein position 120 of SEQ ID No. 1 is not a cysteine.

7. The composition of claim 1, wherein the catalytically-inactive dsPTP is a polypeptide comprising a portion that is at least about 95% identical to SEQ ID No. 1, wherein position 120 of SEQ ID No. 1 is not a cysteine.

8. The composition of claim 1, wherein the catalytically-inactive dsPTP is selected from the group consisting of:
a polypeptide comprising SEQ ID No. 5;
a polypeptide comprising a portion that is at least about 80% identical to SEQ ID No. 5, wherein position 1421 of SEQ ID No. 5 is not a cysteine;
a polypeptide comprising a variant of SEQ ID No. 5 that consists of an N-terminal truncation of SEQ ID No. 5, a C-terminal truncation of SEQ ID No. 5, and any combination thereof;
wherein the polypeptide has about the same binding affinity as SEQ ID No. 5.

9. The composition of claim 1, wherein the matrix comprises a material selected from the group consisting of polystyrene, polysaccharide, agarose, cellulose, polyacrylamide, silica, and porous glass.

10. The composition of claim 1, wherein the catalytically-inactive dsPTP is a polypeptide comprising a variant of SEQ ID No. 1 consisting of an N-terminal truncation of SEQ ID No. 1, a C-terminal truncation of SEQ ID No. 1, or a combination thereof, wherein the N-terminal truncation, the C-terminal truncation or the combination thereof comprises a truncation of a total of about 10 amino acids.

11. The composition of claim 10, wherein the N-terminal truncation, the C-terminal truncation or the combination thereof comprises a truncation of a total of about 30 amino acids.

12. The composition of claim 11, wherein the N-terminal truncation, the C-terminal truncation or the combination thereof comprises a truncation of a total of about 50 amino acids.

13. The composition of claim 12, wherein the N-terminal truncation, the C-terminal truncation or the combination thereof comprises a truncation of a total of about 100 amino acids.

14. The composition of claim 13, wherein the N-terminal truncation, the C-terminal truncation or the combination thereof comprises a truncation of a total of about 135 amino acids.

15. The composition of claim 14, wherein the N-terminal truncation is a total of about 100 amino acids and the C-terminal truncation is a total of about 35 amino acids.

16. The composition of claim 1, further comprising a second affinity capture molecule, wherein the first and second affinity capture molecules are not identical.

17. The composition of claim 16, wherein the second affinity capture molecule is a second catalytically-inactive dsPTP.

18. The composition of claim 17, wherein the second catalytically-inactive dsPTP is selected from the group consisting of:
a polypeptide comprising SEQ ID No. 1;
a polypeptide comprising a portion that is at least about 80% identical to SEQ ID No. 1, wherein position 120 of SEQ ID No. 1 is not a cysteine; and
a polypeptide comprising a portion that is a variant of SEQ ID No. 1 comprising an N-terminal truncation of SEQ ID No. 1, a C-terminal truncation of SEQ ID No. 1, or a combination thereof; wherein the polypeptide has about the same binding affinity as SEQ ID No. 1.

19. A method of removing a phosphorylated polypeptide from a sample, the method comprising:
contacting a sample comprising a phosphorylated polypeptide with the composition of claim 1 under conditions allowing binding of the phosphorylated polypeptide to the affinity capture molecule to form bound phosphorylated polypeptide, and
separating the bound phosphorylated polypeptide from the material that did not bind to the affinity capture molecule, thereby removing a phosphorylated polypeptide from a sample.

20. The method of claim 19, further comprising collecting the material that did not bind to the affinity capture molecule.

21. The method of claim 19, further comprising releasing the bound phosphorylated polypeptide from the affinity capture molecule.

22. The method of claim 19, wherein the catalytically-inactive dsPTP is selected from the group consisting of:
a polypeptide comprising SEQ ID No.1;
a polypeptide comprising a portion that is at least about 80% identical to SEQ ID No. 1, wherein position 120 of SEQ ID No. 1 is not a cysteine;
a polypeptide comprising a portion that is at least about 80% identical to SEQ ID No. 1, wherein position 120 of SEQ ID No. 1 is a cysteine and position 89 of SEQ ID No. 1 is not an aspartic acid, and
a polypeptide comprising a variant of SEQ ID No. 1 that consists of an N-terminal truncation of SEQ ID No. 1, a C-terminal truncation of SEQ ID No. 1, and any combination thereof;
wherein the polypeptide has about the same binding affinity as SEQ ID No. 1.

23. The method of claim 19, wherein the catalytically-inactive dsPTP is a polypeptide comprising a portion that is at least about 90% identical to SEQ ID No. 1, wherein position 120 of SEQ ID No. 1 is not a cysteine.

24. The method of claim 23, wherein the catalytically-inactive dsPTP is a polypeptide comprising a portion that is at least about 95% identical to SEQ ID No. 1, wherein position 120 of SEQ ID No. 1 is not a cysteine.

25. The method of claim 19, wherein the catalytically-inactive dsPTP is selected from the group consisting of:
a polypeptide comprising SEQ ID No. 5;
a polypeptide comprising a portion that is at least about 80% identical to SEQ ID No. 5, wherein position 1421 of SEQ ID No. 5 is not a cysteine;
a polypeptide comprising a variant of SEQ ID No. 5 that consists of an N-terminal truncation of SEQ ID No. 5, a C-terminal truncation of SEQ ID No. 5, and any combination thereof;
wherein the polypeptide has about the same binding affinity as SEQ ID No. 5.

26. The method of claim 19, wherein the matrix comprises a material selected from the group consisting of polystyrene, polysaccharide, agarose, cellulose, polyacrylamide, silica, and porous glass.

27. The method of claim 19, wherein the catalytically-inactive dsPTP is a polypeptide comprising a variant of SEQ ID No. 1 consisting of an N-terminal truncation of SEQ ID No. 1, a C-terminal truncation of SEQ ID No. 1, or a combination thereof, wherein the N-terminal truncation, the C-terminal truncation or the combination thereof comprises a truncation of a total of about 10 amino acids.

28. The method of claim 27, wherein the N-terminal truncation, the C-terminal truncation or the combination thereof comprises a truncation of a total of about 50 amino acids.

29. The method of claim 19, wherein the composition is part of a chromatography column, a spin column, a filter, or a microfluidic device.

30. The method of claim 19, wherein prior to contacting step, the sample is treated to remove highly abundant proteins.

31. A kit for isolating a phosphorylated polypeptide, the kit comprising the composition of claim 1, and instructional material.

32. A kit for isolating a phosphorylated polypeptide, the kit comprising the composition of claim 16, and instructional material.


SEQUENCE LISTING (7 sequences)

SEQ ID NO: 1 - protein, 223 aa, Mus musculus
MEDVKLEFPSVPQCKDDAEEWTYPMRREMQEVLPGLFLGPYSSAMKSKLPILQKHGITHIICIRQNIEANFIKPNFQQLFRYLVLDIADNPVENIIRFFPMTKEFIDGSLQNGGKVLVHGNAGISRSAAFVIAYIMETFGMKYRDAFAYVQERRFCINPNAGFVHQLQEYEAIYLAKLTIQMMSPLQIERSLAVHSGTTGSVKRTHEEDDDFGNMQVATAQNG

SEQ ID NO: 2 - DNA, 672 bp, Mus musculus
atggaggacgtgaaactggagttcccctccgtcccgcagtgcaaagacgacgccgaggagtggacttaccctatgagacgagagatgcaggaagttttgcctggattgttcttaggcccatactcttctgctatgaaaagcaagctacctatacttcagaaacatggcataactcatataatttgcatacggcaaaatattgaagcaaactttattaaaccaaactttcaacaattatttagatatttagttttggatattgctgataatccagttgaaaacataatacgttttttccctatgactaaagagtttattgatggaagcttacaaaatggaggaaaagttcttgtccatgggaatgcaggtatctctagaagtgctgcctttgtcattgcatacatcatggagacgtttgggatgaaatacagagatgcatttgcttatgtccaagaaaggagattttgtattaatcctaatgctggatttgtccaccaacttcaggaatatgaagccatctacttagcaaaattaacaatacagatgatgtcaccactccaaatagaaaggtccttagctgttcattctggtaccacaggtagtgtgaagagaacacatgaggaagatgacgactttggaaacatgcaagtggcaaccgcacagaacggctga

SEQ ID NO: 3 - protein, 223 aa, Homo sapiens
MEDVKLEFPSLPQCKEDAEEWTYPMRREMQEILPGLFLGPYSSAMKSKLPVLQKHGITHIICIRQNIEANFIKPNFQQLFRYLVLDIADNPVENIIRFFPMTKEFIDGSLQMGGKVLVHGNAGISRSAAFVIAYIMETFGMKYRDAFAYVQERRFCINPNAGFVHQLQEYEAIYLAKLTIQMMSPLQIERSLSVHSGTTGSLKRTHEEEDDFGTMQVATAQNG

SEQ ID NO: 4 - DNA, 672 bp, Homo sapiens
atggaggacgtgaagctggagttcccttcccttccacagtgcaaggaagacgccgaggagtggacctaccctatgagacgagagatgcaggaaattttacctggattgttcttaggcccatattcatctgctatgaaaagcaagctacctgtactacagaaacatggaataacccatataatatgcatacgacaaaatattgaagcaaactttattaaaccaaactttcagcagttatttagatatttagtcctggatattgcagataatccagttgaaaatataatacgttttttccctatgactaaggaatttattgatgggagcttacaaatgggaggaaaagttcttgtgcatggaaatgcagggatctccagaagtgcagcctttgttattgcatacattatggaaacatttggaatgaagtacagagatgcttttgcttatgttcaagaaagaagattttgtattaatcctaatgctggatttgtccatcaacttcaggaatatgaagccatctacctagcaaaattaacaatacagatgatgtcaccactccagatagaaaggtcattatctgttcattctggtaccacaggcagtttgaagagaacacatgaagaagaggatgattttggaaccatgcaagtggcgactgcacagaatggctga

SEQ ID NO: 5 - protein, 1867 aa, Homo sapiens
MARLADYFVLVAFGPHPRGSGEGQGQILQRFPEKDWEDNPFPQGIELFCQPSGWQLCPERNPPTFFVAVLTDINSERHYCACLTFWEPAEPSQETTRVEDATEREEEGDEGGQTHLSPTAPAPSAQLFAPKTLVLVSRLDHTEVFRNSLGLIYAIHVEGLNVCLENVIGNLLTCTVPLAGGSQRTISLGAGDRQVIQTPLADSLPVSRCSVALLFRQLGITNVLSLFCAALTEHKVLFLSRSYQRLADACRGLLALLFPLRYSFTYVPILPAQLLEVLSTPTPFIIGVNAAFQAETQELLDVIVADLDGGTVTIPECVHIPPLPEPLQSQTHSVLSMVLDPELELADLAFPPPTTSTSSLKMQDKELRAVFLRLFAQLLQGYRWCLHVVRIHPEPVIRFHKAAFLGQRGLVEDDFLMKVLEGMAFAGFVSERGVPYRPTDLFDELVAHEVARMRADENHPQRVLRHVQELAEQLYKNENPYPAVAMHKVQRPGESSHLRRVPRPFPRLDEGTVQWIVDQAAAKMQGAPPAVKAERRTTVPSGPPMTAILERCSGLHVNSARRLEVVRNCISYVFEGKMLEAKKLLPAVLRALKGRAARRCLAQELHLHVQQNRAVLDHQQFDFVVRMMNCCLQDCTSLDEHGIAAALLPLVTAFCRKLSPGVTQFAYSCVQEHVVWSTPQFWEAMFYGDVQTHIRALYLEPTEDLAPAQEVGEAPSQEDERSALDVASEQRRLWPTLSREKQQELVQKEESTVFSQAIHYANRMSYLLLPLDSSKSRLLRERAGLGDLESASNSLVTNSMAGSVAESYDTESGFEDAETCDVAGAVVRFINRFVDKVCTESGVTSDHLKGLHVMVPDIVQMHIETLEAVQRESRRLPPIQKPKLLRPRLLPGEECVLDGLRVYLLPDGREEGAGGSAGGPALLPAEGAVFLTTYRVIFTGMPTDPLVGEQVVVRSFPVAALTKEKRISVQTPVDQLLQDGLQLRSCTFQLLKMAFDEEVGSDSAELFRKQLHKLRYPPDIRATFAFTLGSAHTPGRPPRVTKDKGPSLRTLSRNLVKNAKKTIGRQHVTRKKYNPPSWEHRGQPPPEDQEDEISVSEELEPSTLTPSSALKPSDRMTMSSLVERACCRDYQRLGLGTLSSSLSRAKSEPFRISPVNRMYAICRSYPGLLIVPQSVQDNALQRVSRCYRQNRFPVVCWRSGRSKAVLLRSGGLHGKGVVGLFKAQNAPSPGQSQADSSSLEQEKYLQAVVSSMPRYADASGRNTLSGFSSAHMGSHGKWGSVRTSGRSSGLGTDVGSRLAGRDALAPPQANGGPPDPGFLRPQRAALYILGDKAQLKGVRSDPLQQWELVPIEVFEARQVKASFKKLLKACVPGCPAAEPSPASFLRSLEDSEWLIQIHKLLQVSVLVVELLDSGSSVLVGLEDGWDITTQVVSLVQLLSDPFYRTLEGFRLLVEKEWLSFGHRFSHRGAHTLAGQSSGFTPVFLQFLDCVHQVHLQFPMEFEFSQFYLKFLGYHHVSRRFRTFLLDSDYERIELGLLYEEKGERRGQVPCRSVWEYVDRLSKRTPVFHNYMYAPEDAEVLRPYSNVSNLKVWDFYTEETLAEGPPYDWELAQGPPEPPEEERSDGGAPQSRRRVVWPCYDSCPRAQPDAISRLLEELQRLETELGQPAERWKDTWDRVKAAQRLEGRPDGRGTPSSLLVSTAPHHRRSLGVYLQEGPVGSTLSLSLDSDQSSGSTTSGSRQAARRSTSTLYSQFQTAESENRSYEGTLYKKGAFMKPWKARWFVLDKTKHQLRYYDHRVDTECKGVIDLAEVEAVAPGTPTMGAPKTVDEKAFFDVKTTRRVYNFCAQDVPSAQQWVDRIQSCLSDA

SEQ ID NO: 6 - DNA, 5604 bp, Homo sapiens
atggcgcggctcgcggactacttcgtgctggtggcgttcgggccgcacccgcgcgggagtggggaaggccagggccagattctgcagcgcttcccagagaaggactgggaggacaacccattcccccagggcatcgagctgttttgccagcccagcgggtggcagctgtgtcccgagaggaatccaccgaccttctttgttgctgtcctcaccgacatcaactccgagcgccactactgcgcctgcttgaccttctgggagccagcggagccttcacaggaaacgacgcgcgtggaggatgccacagagagggaggaagagggggatgagggaggccagacccacctgtctcccacagcacctgccccatctgcccagctgtttgcaccgaagacgctggtactggtgtcgcgactcgaccacacggaggtgttcaggaacagccttggcctcatctatgccatccacgtggagggcctgaatgtgtgcctggagaacgtgattgggaacctgctgacgtgcactgtgcccctggctgggggctcgcagaggacgatctctttgggggctggtgaccggcaggtcatccagactccactggccgactcgctgcccgtcagccgctgcagcgtggccctgctcttccgccagctaggcatcaccaacgtgctgtctttgttctgtgccgccctcacggagcacaaggttctcttcctgtcccggagctaccagcggctcgccgatgcctgtaggggcctcctggcactgctgtttcctctcagatacagcttcacctatgtgcccatcctgccggctcagctgctggaggtcctcagcacacccacgcccttcatcattggggtcaacgcggccttccaggcagagacccaggagctgctcgatgtgattgttgctgatctggatggagggacggtcaccattcctgagtgtgtgcacattccacccttgccagagccactgcagagtcagacgcacagtgtgctgagcatggtcctggacccggagctggagttggctgacctcgccttccctccgcccacgacatccacctcctccctgaagatgcaggacaaggagctgcgcgcggtcttcctgcggctgttcgctcagctgctgcagggctatcgctggtgcctgcacgtcgtgcgcatccacccggagcctgtcatccgcttccataaggcagccttcctgggccagcgtgggctggtagaggacgatttcctgatgaaggtgctggagggcatggcctttgctggctttgtgtcagagcgtggggtcccataccgccctacggacctgttcgatgagctggtggcccacgaggtggcaaggatgcgggcggatgagaaccacccccagcgtgtcctgcgtcacgtccaggaactggcagagcagctctacaagaacgagaacccgtacccagccgtggcgatgcacaaggtacagaggcccggtgagagcagccacctgcgacgggtgccccgacccttcccccggctggatgagggcaccgtgcagtggatcgtggaccaggctgcagccaagatgcagggtgcacccccagctgtgaaggccgagaggaggaccaccgtgccctcagggccccccatgactgccatactggagcggtgcagtgggctgcatgtcaacagcgcccggcggctggaggttgtgcgcaactgcatctcctacgtgtttgaggggaaaatgcttgaggccaagaagctgctcccagccgtgttgagggccctgaaggggcgagctgcccgccgctgcctcgcccaggagctgcacctgcatgtgcagcagaaccgtgcggtcctggaccaccagcagtttgactttgtcgtccgtatgatgaactgctgcctgcaggactgcacttctctggacgagcatggcattgcggcggctctgctgcctctggtcacagccttctgccggaagctgagcccgggggtgacgcagtttgcatacagctgtgtgcaggagcacgtggtgtggagcacgccacagttctgggaggccatgttctatggggatgtgcagactcacatccgggccctctacctggagcccacggaggacctggcccccgcccaggaggttggggaggcaccttcccaggaggacgagcgctctgccctagacgtggcttctgagcagcggcgcttgtggccaactctgagtcgtgagaagcagcaggagctggtgcagaaggaggagagcacggtgttcagccaggccatccactatgccaaccgcatgagctacctcctcctgcccctggacagcagcaagagccgcctacttcgggagcgtgccgggctgggcgacctggagagcgccagcaacagcctggtcaccaacagcatggctggcagtgtggccgagagctatgacacggagagcggcttcgaggatgcagagacctgcgacgtagctggggctgtggtccgcttcatcaaccgctttgtggacaaggtctgcacggagagtggggtcaccagcgaccacctcaaggggctgcatgtcatggtgccagacattgtccagatgcacatcgagaccctggaggccgtgcagcgggagagccggaggctgccgcccatccagaagcccaagctgctgcggccgcgcctgctgccgggtgaggagtgtgtgctggacggcctgcgcgtctacctgctgccggatgggcgtgaggagggcgcggggggcagtgctgggggaccagcattgctcccagctgagggcgccgtcttcctcaccacgtaccgggtcatcttcacggggatgcccacggaccccctggttggggagcaggtggtggtccgctccttcccggtggctgcgctgaccaaggagaagcgcatcagcgtccagacccctgtggaccagctcctgcaggacgggctccagctgcgctcctgcacattccagctgctgaaaatggcctttgacgaggaggtggggtctgacagcgccgagctcttccgtaagcagctgcataagctgcggtacccgccggacatcagggccacctttgcgttcaccttgggctctgcccacacacctggccggccaccgcgagtcaccaaggacaagggtccttccctcagaaccctgtcccggaacctggtcaagaacgccaagaagaccatcgggcggcagcatgtcactcgcaagaagtacaacccccccagctgggagcaccggggccagccgccccctgaggaccaggaggacgagatctcagtgtcggaggagctggagcccagcacgctgaccccgtcctcagccctgaagccctccgaccgcatgaccatgagcagcctggtggaaagggcttgctgtcgcgactaccagcgcctcggtctgggcaccctgagcagcagcctgagccgggccaagtctgagcccttccgcatttctccggtcaaccgcatgtatgccatctgccgcagctacccagggctgctgatcgtgccccagagtgtccaggacaacgccctgcagcgcgtgtcccgctgctaccgccagaaccgcttccccgtggtctgctggcgcagcgggcggtccaaggcggtgctgctgcgctctggaggcctgcatggcaaaggtgtcgtcggcctcttcaaggcccagaacgcaccttctccaggccagtcccaggcggactcgagtagcctggagcaggagaagtacctgcaggctgtggtcagctccatgccccgctacgccgacgcgtcgggacgcaacacgcttagcggcttctcctcagcccacatgggcagtcacggtaagtggggcagtgtccggaccagtggacgcagcagtggccttggcaccgatgtgggctcccggctagctggcagagacgcgctggccccaccccaggccaacgggggccctcccgacccgggcttcctgcgtccgcagcgagcagccctctatatccttggggacaaagcccagctcaagggtgtgcggtcagaccccctgcagcagtgggagctggtgcccattgaggtattcgaggcacggcaggtgaaggctagcttcaagaagctgctgaaagcatgtgtcccaggctgccccgctgctgagcccagcccagcctccttcctgcgctcactggaggactcagagtggctgatccagatccacaagctgctgcaggtgtctgtgctggtggtggagctcctggattcaggctcctccgtgctggtgggcctggaggatggctgggacatcaccacccaggtggtatccttggtgcagctgctctcagaccccttctaccgcacgctggagggctttcgcctgctggtggagaaggagtggctgtccttcggccatcgcttcagccaccgtggagctcacaccctggccgggcagagcagcggcttcacacccgtcttcctgcagttcctggactgcgtacaccaggtccacctgcagttccccatggagtttgagttcagccagttctacctcaagttcctcggctaccaccatgtgtcccgccgtttccggaccttcctgctcgactctgactatgagcgcattgagctggggctgctgtatgaggagaagggggaacgcaggggccaggtgccgtgcaggtctgtgtgggagtatgtggaccggctgagcaagaggacgcctgtgttccacaattacatgtatgcgcccgaggacgcagaggtcctgcggccctacagcaacgtgtccaacctgaaggtgtgggacttctacactgaggagacgctggccgagggccctccctatgactgggaactggcccaggggccccctgaacccccagaggaagaacggtctgatggaggcgctccccagagcaggcgccgcgtggtgtggccctgttacgacagctgcccgcgggcccagcctgacgccatctcacgcctgctggaggagctgcagaggctggagacagagttgggccaacccgctgagcgctggaaggacacctgggaccgggtgaaggctgcacagcgcctcgagggccggccagacggccgtggcacccctagctccctccttgtgtccaccgcaccccaccaccgtcgctcgctgggtgtgtacctgcaggaggggcccgtgggctccaccctgagcctcagcctggacagcgaccagagtagtggctcaaccacatccggctcccgtcaggctgcccgccgcagcaccagcaccctgtacagccagttccagacagcagagagtgagaacaggtcctacgagggcactctgtacaagaagggggccttcatgaagccttggaaggcccgctggttcgtgctggacaagaccaagcaccagctgcgctactacgaccaccgtgtggacacagagtgcaagggtgtcatcgacttggcggaggtggaggctgtggcacctggcacgcccactatgggtgcccctaagactgtggacgagaaggccttctttgacgtgaagacaacgcgtcgcgtttacaacttctgtgcccaggacgtgccctcggcccagcagtgggtggaccggatccagagctgcctgtcggacgcctga

SEQ ID NO: 7 - protein, 9 aa, Artificial sequence
  Catalytic motif for protein tyrosine phosphatases
  MISC_FEATURE (3)..(4): Xaa can be any naturally occurring amino acid
  MISC_FEATURE (6)..(7): Xaa can be any naturally occurring amino acid
  MISC_FEATURE (9)..(9): Xaa can be Ser or Thr
HCXXGXXRX